(19) United States
(12) Patent Application Publication
(10) Pub. No.: US 2024/0389853 A1
(43) Pub. Date: Nov. 28, 2024

(54) DEVICE AND METHODS FOR OCULAR MOTOR TESTING

(71) Applicant: The Children’s Mercy Hospital, Kansas City, MO (US)

(72) Inventor: Andrea Thorne, Kansas City, MO (US)

(21) Appl. No.: 18/796,637

(22) Filed: Aug. 7, 2024

Related U.S. Application Data
(63) Continuation-in-part of application No. PCT/US2023/068278, filed on Jun. 12, 2023.
(60) Provisional application No. 63/353,122, filed on Jun. 17, 2022.

(51) Int. Cl. A61B 3/113; A61B 5/00

(52) U.S. Cl. CPC A61B 3/113; A61B 5/4076

(57) ABSTRACT

A compact tool (10) for assessing eye tracking measures that can be used for ocular motor testing of an individual under a variety of circumstances involving neurological impairment. The tool comprises an elongated shaft (12) and a slide member (26) that is adapted to slide along the shaft. The tool can be used to perform one or more eye movement assessments, including smooth pursuit assessment, a convergence assessment, a saccades assessment, and a vestibular ocular reflex assessment, or assess pupillary response.

### CROSS-REFERENCE TO RELATED APPLICATIONS

This application is a continuation in part of International Patent Application No. PCT/US2023/068278, filed Jun. 12, 2023, which claims the benefit of U.S. Provisional Patent Application No. 63/353,122, filed Jun. 17, 2022. Both of which are incorporated by reference herein in their entireties.

### BACKGROUND OF THE INVENTION

### Field of the Invention

Embodiments of the present invention relate to a compact tool for assessing eye tracking measures that can be used for ocular motor testing of an individual under a variety of circumstances involving neurological impairment. Examples of these impairments include a suspected concussion, acute neurological impairment, intoxication, etc. that may impair visual tracking post injury.

### Description of the Prior Art

Ocular evaluations of patients with suspected and/or known neurologic impairments currently lack a standardized method of assessment. Many new products are being produced utilizing artificial intelligence (AI) and other electronic means of measurement; however an assessment tool that is easily portable and does not require electronic means, or internet connectivity to assess the four major vestibular ocular motor skills remains to be established. These assessments are needed for varying neurologic conditions, and in particular, concussion.

There is a growing awareness of the long-term adverse health effects of repeated head trauma, such as can be sustained in sporting activities. Thus, healthcare professionals have made it a point of emphasis to identify suspected concussion and hold the patient out of further activity until the concussion can be confirmed by a health care professional. Diagnosis of concussion can be a subjective exercise that often relies upon the skill and expertise of the medical professional observing ocular motion. Medical professionals often utilize various cognitive and motor function tests to evaluate brain function before and after a head injury, and based upon the results of those tests, render a diagnosis.

The ocular motor tests are not standardized within the medical profession with a specific tool and can be conducted using a variety of assessment tools. The lack of standardized testing can lead to imprecise diagnoses of concussion and other neurologic conditions, which increases the chance that a neurologically impaired patient could be cleared to resume activity, or that a neuro-intact patient will be needlessly held out from activity for a period of time.

Thus, a need exists in the art for a standardized ocular motor assessment tool that can be used to perform a variety of assessments in order to screen for neuro-deficits more precisely in patients suspected of suffering impairment.

As previously discussed, ocular motor testing can be used in other applications beyond assessment of traumatic injuries. For example, ocular motor testing may be used to screen for other vestibular deficits that may be a result of a congenital defect or intoxication. Particularly, alcohol intoxication can result in vestibular deficits that can be detected through ocular motor assessment. A standardized tool that can be used by law enforcement or other first responders would be helpful in assessing impairment of an individual suspected of, for example, operating a motor vehicle while intoxicated.

### SUMMARY OF THE INVENTION

According to one embodiment of the present invention there is provided an ocular motor assessment tool comprising a graduated, elongated shaft. The elongated shaft comprises a proximal end that is adapted to be placed into contact with the patient's nose during use. The tool further comprises a slide member that is adapted to slide along at least a portion of the length of the elongated shaft. The position of the slide member on the shaft is recorded by the location of the slide member relative to the shaft graduations. A tab is connected to and slidable with the slide member. The tab is configured to extend upwardly from the slide member. When the proximal end is placed in contact with the patient's nose and the shaft is extended horizontally away from the patient's head, the tab has a surface that is observable by the patient. The tab surface comprises a first focal character located approximately level to the eyes of the patient.

According to a further embodiment of the present invention there is provided an ocular motor assessment tool comprising a graduated, elongated shaft having a proximal end configured to be placed into contact with a nose of a patient and a distal end. A light assembly is provided that comprises a light source that is operable to emit a beam of light from the elongated shaft. A slide member is also provided that is adapted to slide along at least a portion of the length of the elongated shaft. The position of the slide member on the shaft is recorded by the location of the slide member relative to the shaft graduations.

According to another embodiment of the present invention there is provided a method of ocular motor screening of a patient, especially when screening for possible concussion. The method comprises first providing an ocular motor assessment tool according to any embodiment described herein. Preferably, the assessment tool is then extended from a collapsed configuration to an extended configuration. Then, one or more eye movement assessments are performed with the assessment tool selected from the group consisting of a smooth pursuit assessment, a convergence assessment, saccades assessment, and a vestibular ocular reflex assessment.

### BRIEF DESCRIPTION OF THE DRAWINGS

FIG. 1 is a perspective view of an ocular motor assessment tool in a collapsed configuration;

FIG. 2 is an alternate perspective view of the tool of FIG. 1 in the collapsed configuration;

FIG. 3 is a perspective view of the tool of FIG. 1 in an extended configuration;

FIG. 4 is an alternate perspective view of the tool of FIG. 1 in the extended position;

FIG. 5 is a side view of the tool of FIG. 1 in the extended position and being used in performing an assessment of convergence;

FIG. 6 is a perspective view of the tool in FIG. 1 in the extended position and being used in performing an assessment of convergence;

FIG. 7 is a perspective view of an alternate embodiment of an ocular motor assessment tool comprising a light assembly configured to emit a beam of light; and

FIG. 8 is a partial cross-sectioned view of the tool of FIG. 7 showing the light assembly installed within the tool's elongated shaft.

While the drawings do not necessarily provide exact dimensions or tolerances for the illustrated components or structures, the drawings are to scale with respect to the relationships between the components of the structures illustrated in the drawings.

### DETAILED DESCRIPTION OF THE PREFERRED EMBODIMENT

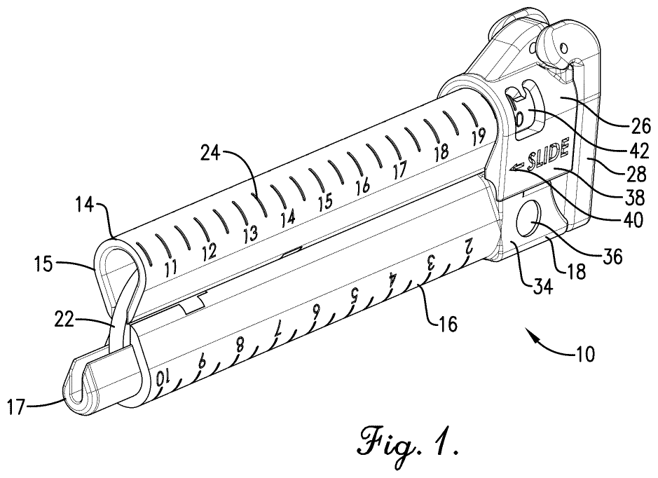

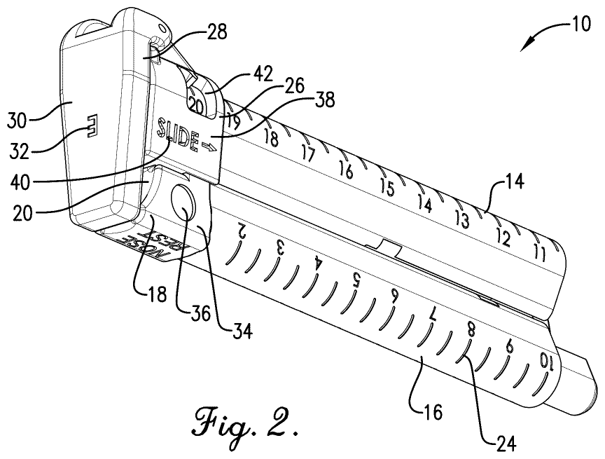

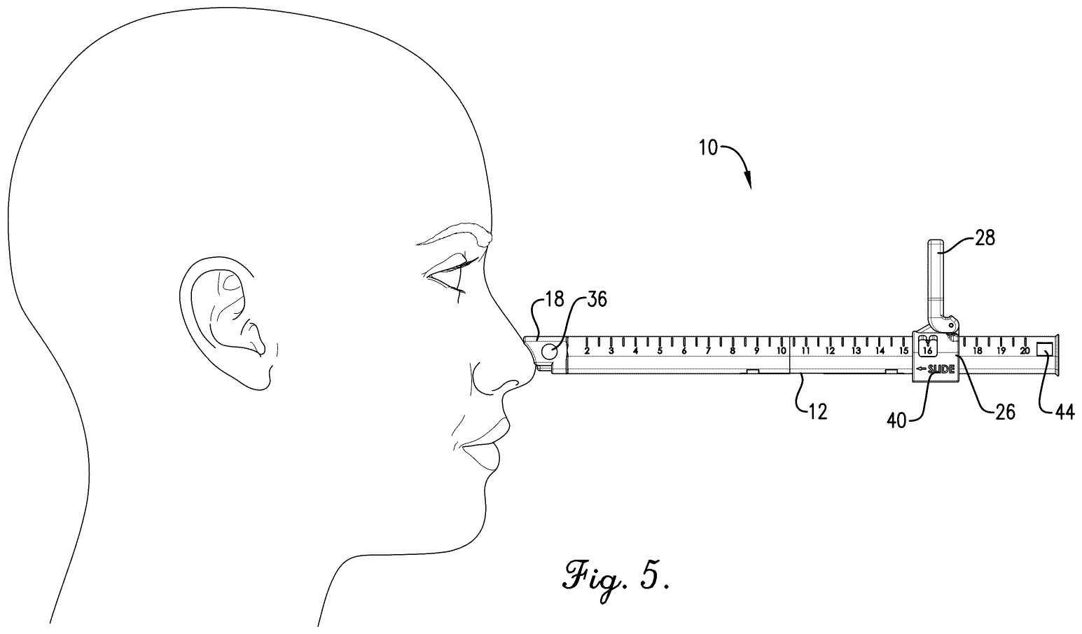

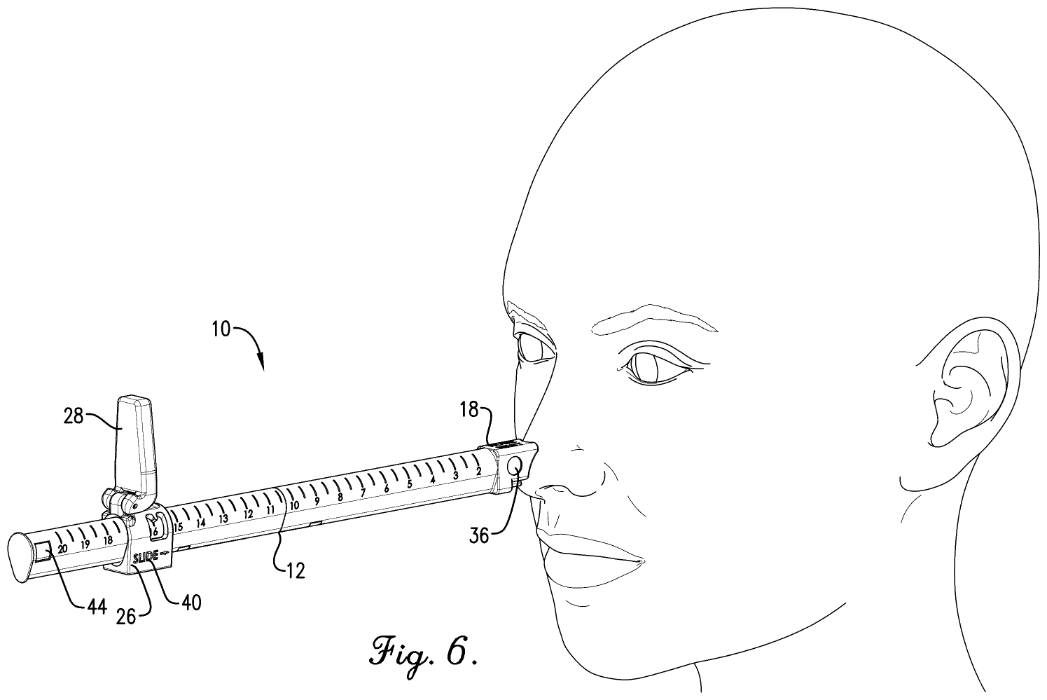

With reference to FIGS. 1-6, an ocular motor assessment tool 10 is illustrated. The tool 10 comprises an elongated shaft 12. In one or more preferred embodiments, the elongated shaft comprises at least two collapsible segments 14, 16. However, it is within the scope of the present invention for the shaft to be of a one-piece, unitary construction. In preferred embodiments, the tool also comprises a nose piece 18 that includes at least one contoured surface 20 that is configured to conform to the nose of a patient, especially as seen in FIGS. 5 and 6. However, elongated shaft 12 may simply be provided with a blunt end that is configured to be placed into engagement with the patient's nose. The segments 14, 16 may be connected to each other by a tether 22, which may be an elastic cord (or other material), so that the two segments are secured together at all times. Alternatively, segments 14, 16 may be detachable from each other for storage when the tool 10 is not in use. The segments 14, 16 may be equipped with mating ends 15, 17, respectively, for assisting with securing the segments together. For example, as shown, mating ends 15, 17 may comprise male and female connecting structures configured to be frictionally secured together. Alternatively, ends 15, 17 may comprise threaded connections or spring-loaded pin and hole connections, for example. The elongated shaft 12 can be graduated to denote distance from the patient's nose when the tool is in use. The graduations 24 can be in the form of tick marks, which may include numerical values assigned to some or all of the tick marks, preferably in centimeters.

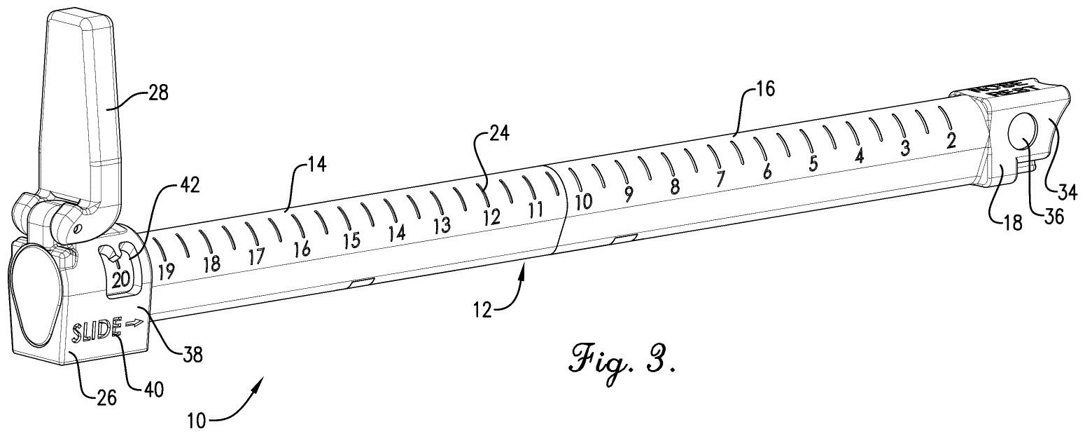

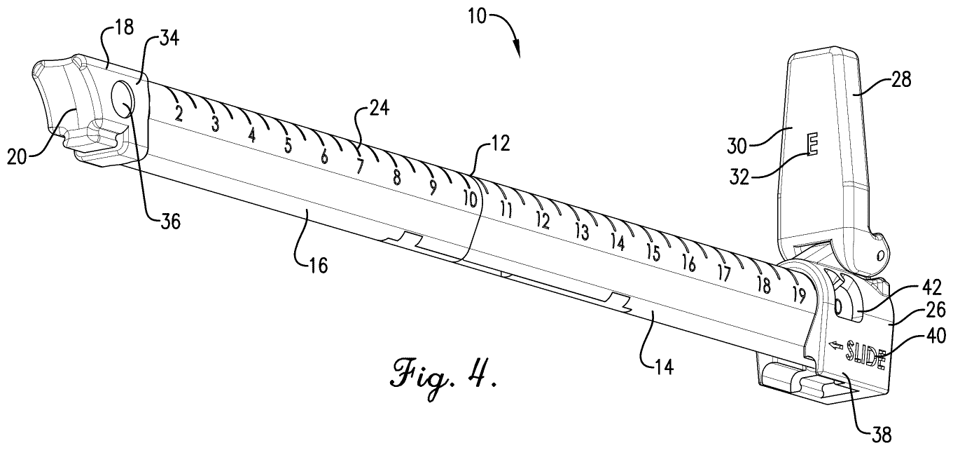

The tool 10 further includes a slide member 26 that is adapted to slide along at least a portion of the length of the elongated shaft 12. The position of the slide member 26 on the shaft 12 is indicated by the location of the slide member 26 relative to the shaft graduations 24. The tool 10 further comprises a tab 28 that is connected to and slidable with the slide member 26. The tab 28 is configured to extend upwardly from the slide member 28 as shown in FIGS. 3 and 4. The tab 28 has a tab surface 30 that comprises a first focal character 32 located thereon. The focal characters described herein may comprise any distinctive visual indicator that can be observed by the patient during usage of the tool 10. In certain embodiments, the focal characters may comprise letters, numbers, symbols, shapes, objects, images, colors, patterns, or any combination thereof. The focal characters should be selected so that they are easily identifiable in relation to other structures of tool 10, and in certain embodiments, are easily distinguishable from each other. In one or more embodiments, the focal characters described herein have font sizes of 12 to 16 points, preferably 14 points (with one point equaling 1/72 inch).

In certain embodiments, the slide member 26 includes a window 42 through which the shaft graduations 24 can be viewed.

The nose piece 18 includes a sidewall surface 34 that is laterally disposed from the contoured surface 20 and has a second focal character 36 located thereon. As depicted in FIGS. 1-6, the second focal character 36 comprises a circle; however, this character could be any type of character as described above. In certain embodiments, as can be seen in FIG. 5, a third focal character 44 can be provided at the opposite end from the second focal character 36, i.e., at the distal end of device 10. The third focal character 44 is shown as comprising a square, but it could be of any type of focal character described above. Preferably, the second and third focal characters 36, 40 are located on the same general side of the tool 10 so that they can be viewed simultaneously by a user without having to re-orient the tool within the user's field of view. Also, it is preferred for the second and third focal characters 36, 44 to be complementary to each other, i.e., both shapes, both letters, both numbers, etc., but such need not always be the case. Also, the two focal characters preferably are different from each other to help explain the testing to be performed to the patient (i.e., square and a circle, the numbers three and five (non-repeating characters)).

In addition, the slide member 26 comprises a sidewall surface 38 that comprises yet another focal character 40 located thereon. As depicted, focal character 40 comprises an arrow and the word “slide.” In addition to providing a simple instruction on device operation, focal character 40 could be used in the same manner as the third focal character 44, as slide member 26, when placed in the most distal position, could obscure third focal character 44. Thus, focal character 40 could be any type of focal character described above.

The tool is shiftable between a collapsed configuration, as shown in FIG. 1, and an extended configuration, as shown in FIG. 3. When in the collapsed configuration, the slide member 26 is positioned adjacent to the nose piece 18. In certain embodiments, the slide member 26 can be adapted to engage and/or interlock with the nose piece 18 to inhibit relative movement between the two segments 14, 16 when in the collapsed configuration. The two segments 14, 16 can also be configured such that when in the collapsed configuration one segment is telescopically received within another of the segments.

In certain embodiments, when the contoured surface 20 is placed in contact with the patient's nose and the shaft 12 is extended horizontally away from the patient's head, as shown in FIGS. 5 and 6, the tab surface 30 and the first focal character 32 are in facing relationship to the patient's face with the first focal character 32 being located approximately level to, or slightly below, the eyes of the patient. In the extended position, the tab 28 extends upwardly from the slide member 26. In the collapsed configuration, the tab 28 is shifted to a stored configuration in which the tab 28 extends toward the nose piece 18.

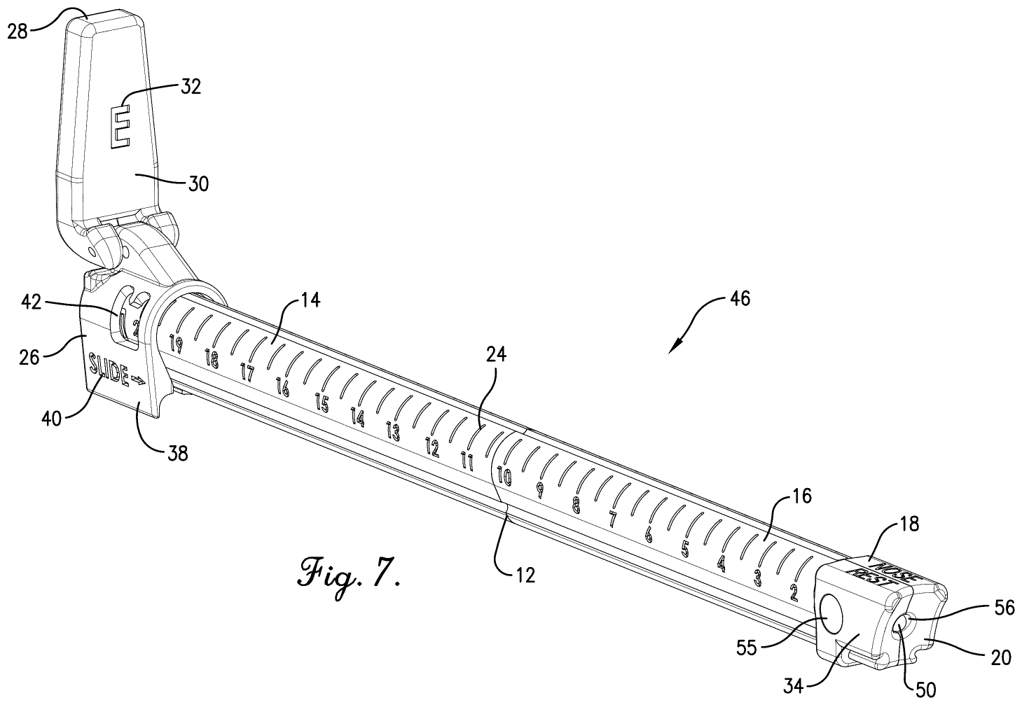

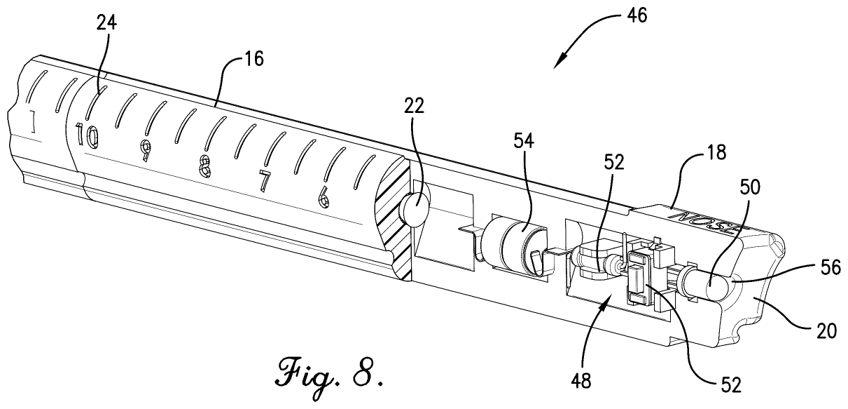

Turning to FIGS. 7 and 8, an alternate ocular motor assessment tool 46 is illustrated. Tool 46 is similar in many respects to tool 10 described above and features present in tool 46 that are shared with tool 10 bear the same reference numerals. However, where tool 46 differs from tool 10 is that tool 46 comprises a light assembly 48 that is configured to emit a beam of light from the tool. As can best be seen in FIG. 8, light assembly 48 comprises a light source 50, such as an LED or light bulb, a switch 52 electrically coupled to and operable to control light source 50, a resistor 52 electrically coupled to switch 52, one or more batteries 54 electrically coupled to resistor 52, and a button 55 (FIG. 7) that acts as a user interface for and is mechanically coupled to switch 52. In certain embodiments, light assembly 48 is located within collapsible segment 16 and proximate to the end comprising nose piece 18; however, this need not always be the case as it is within the scope of the present invention to locate light assembly 48 within collapsible segment 14.

In particular embodiments, nose piece 18 comprises a recess or opening 56 that extends through contoured surface 20 and inside of which light source 50 is located. Recess 56 can be located centrally within nose piece 18 and centered within contoured surface 20. It is preferably that light source 50 not extend beyond contoured surface 20 in order to avoid user discomfort when contoured surface 20 is placed into contact with the user's nose.

In one embodiment, button 55 is of the same shape as the second focal character 36 of tool 10. Thus, tool 46 retains all of the same functionality as tool 10 with the added function of being able to emit a beam of light therefrom. As explained in further detail below, the beam of light that is generated by light source 50 can be used generally to examine a subject's eye. For example, tool 10 can be used to assess pupil response in a subject by shining the beam of light into a subject's eye, to assess jaundice, or bloodshot eyes.

As described in greater detail below, the tool is configured for use in performing one or more eye movement assessments including a smooth pursuit assessment, a convergence assessment, a saccades assessment, and a vestibular ocular reflex (VOR) assessment. Both the saccades and VOR assessments are understood to include horizontal and vertical assessment components. These tests may be performed in the stated order, or in any other order as recommended by a medical professional. In addition to the generalized use of the tool for these assessments, if used at the time of a suspected concussion, these measurements can be used to assist in the screening for concussion and to assess recovery. The tool can also be used to perform baseline assessments on the patient prior to experiencing any head trauma, such that when the patient is suspected of concussion for example, those baseline assessments can be referenced to screen for concussion more accurately. Also, the tool can be used to screen for ocular motor deficits that may be the resultant of any neurological impairments.

Embodiments of the ocular motor assessment tool can be used to perform one or more eye movement assessments to assist in the screening of a vestibular deficit, such as, but not limited to, concussion. These tests include a convergence assessment, a smooth pursuit assessment, a saccades assessment, and a vestibular ocular reflex assessment.

### Assessment of Convergence

Convergence is a visual response elicited by interest in a near object, namely the simultaneous inward movement of both eyes toward each other, usually in an effort to maintain single binocular vision when viewing an object. In the convergence assessment, the appropriate distance from the patient's eyes for the performance of additional assessments is determined. For the convergence test, the slider is set to the furthest graduated mark from the patient's face. In the illustrations, this is the 20 cm mark. The nose piece of the tool is then placed on the nose of the patient as depicted, for example, in FIGS. 5 and 6. The patient may assist with placement and holding of the tool throughout the test. The slider, which comprises an upwardly extending tab having a focal character thereon, is moved slowly toward the patient's face until the patient, while looking at the focal character, reports double vision, blurry vision, or the healthcare provider notes asymmetric movement of the eyes medially. The location of the slider is then recorded using the graduations, preferably in centimeters. In certain embodiments, an abnormal result can be reported if, at the conclusion of the test, the slider is positioned at a graduation that is greater than 6 cm. As depicted, the focal character can be a letter such as a capital “E,” similar to what may appear on a standard vision test chart. However, any focal character (e.g., a letter, number, shape, object, etc.) can be used.

### Assessment of Smooth Pursuit

Smooth pursuit movements are slower tracking movements of the eyes in which the eyes remain fixated on a moving object. The assessment is designed to keep a moving stimulus on the fovea, the area of the retina specialized for high acuity in the center of the macula and contains a high density of cones and few rods. It is one of two ways that visual animals can voluntarily shift gaze. In the smooth pursuit assessment, any of the focal characters located on the device can be used as a moving object that the patient's eyes should be fixated upon while the patient's head remains stationary. The assessment tool is then held away from the patient's face no closer than the converging distance determined in the convergence assessment and with the device midline and at eye level. The assessment tool is then moved horizontally (left and right) in front of the patient, and the full ocular range of motion, dictated by the eyes, is assessed. When at the end range of the left and right, the tool is move superiorly and inferiorly in an “H” motion (to the side, then up and down, then to the opposite side, and then up and down). Optionally, the tool can be moved superiorly and inferiorly in midline to fully assess the ocular range of motion. When at the end of each range of ocular motion, it should be noted if there are small jumps of nystagmus (1-2 beats at the end or range is acceptable). It should be noted if the eyes are moving symmetrically and if they can hold the edge or ocular range. Persisting nystagmus or more than 3 beats of nystagmus constitute a failure for the assessment. It should be noted whether the patient is turning his or her head to complete the symmetrical range of motion. Generally, the assessment is evaluated by noting that the patient's performance was “within normal limits” or “not within normal limits.” The assessment can be repeated until the test administrator is confident in the accuracy of the assessment findings.

### Assessment of Saccades

Saccades are rapid, ballistic movements of the eyes that abruptly change the point of fixation. They range in amplitude from the small movements made while reading, for example, to the much larger movements made while gazing around a room. Saccades can be elicited voluntarily, but occur reflexively whenever the eyes are open, even when fixated on a target. Saccadic eye movements are said to be ballistic because the saccade-generating system cannot respond to subsequent changes in the position of the target during the course of the eye movement. If the target moves again during this time (which is on the order of 15-100 ms), the saccade will miss the target, and a second saccade must be made to correct the error. The saccades assessment can be performed in both horizontal and vertical directions. The second and third focal characters (e.g., the circle and square) are pointed towards the patient. For the horizontal measurement, the extended assessment tool is held in front of the patient's face, with the longitudinal axis of the tool being oriented substantially horizontally, no closer than the converging distance determined in the convergence assessment, and preferably 2 to 3 feet from the patient. The patient is then asked to move between looking at one focal character to the other, while keeping his or her head stationary. The process can be repeated for 2-4 cycles as necessary to allow for the test administrator to have confidence in the findings. The healthcare provider assesses for hypo or hypermetric saccadic movements and records these findings. The vertical saccades assessment is performed similarly, except that the assessment tool is positioned in front of the patient's face with its longitudinal axis oriented substantially vertically. The patient is then asked to move between looking at one focal character to the other. The healthcare provider assesses the patient for hypo or hypermetric saccadic movements and records these findings. The assessment score is reported as being “within normal limits” or “not within normal limits.”

### Assessment of Vestibular Ocular Reflex

The vestibulo-ocular reflex (VOR) is a reflex acting to stabilize gaze during head movement. Eye movement during this reflex is due to activation of the vestibular system. The reflex acts to stabilize images on the retinas of the eye during head movement. Gaze is held steadily on a location by producing eye movements in the direction opposite that of head movement. For example, when the head moves to the right, the eyes move to the left, meaning the image a person sees stays the same even though the head has turned. Since slight head movement is present all the time, VOR is necessary for stabilizing vision. In the VOR assessment, any focal character located on the device can be used for this assessment. The device is positioned at the center of the patient's visual field at eye level at a distance that is no closer than the convergence distance determined during the convergence assessment, and preferably about 2 to 3feet in front of the patient. The device is held statically in front of the patient.

The assessment has two stages: a yaw movement stage and a pitch movement stage. In the yaw movement stage, the patient is asked to focus on the focal character and move his or her head from left to right as if providing a non-verbal “no” motion (i.e., shaking the head from side to side). The frequency of this motion should be approximately 120 beats per minute while focusing on the focal character while the patient is in a standing position. The patient continues this yawing motion until the patient reports experiencing dizziness or loss of balance, or other adverse effect. The healthcare provider records the number of seconds that the patient was able to tolerate the yawing motion before experiencing the adverse effect. In the pitch movement stage, the patient is asked to move his or her head up and down as if providing a non-verbal “yes” motion (i.e., nodding of the head up and down). The frequency of this motion should be approximately 120 beats per minute while focusing on the focal character while the patient is in a standing position. The patient continues this pitching motion until the patient reports experiencing dizziness or loss of balance, or other adverse effect. The healthcare provider records the number of seconds that the patient was able to tolerate the pitching motion before experiencing the adverse effect. The patient is considered to have passed the assessment if each movement stage is tolerated for 120 seconds without a loss of balance.

### Assessment of Pupillary Light Reflex

The pupillary light reflex (PLR) is a reflex that controls the diameter of the pupil in response to light intensity. This reflex assists in the adaptation of vision to various levels of lightness or darkness and is an automatic reflex that adjusts the amount of light that reaches the retina. A greater intensity of light causes the pupil to constrict, thereby allowing less light to reach the retina whereas a lower intensity of light causes the pupil to dilate, thereby allowing more light to reach the retina. The PLR is traditionally assessed using a penlight to test whether a patient has a head injury. The ocular motor assessment tool 46 of FIGS. 7 and 8 can be used to perform the assessment of PLR much in the same way that a clinician might use a traditional pen light.

One simple way to assess whether a patient has dilated pupils is to shine a light from the outer edge of each eye inward. The patient is asked to focus on a distant target while the light is shown independently on each eye. The healthcare provider assesses the patient for pupil constriction response time, and if the pupil without the light shown directly on it constricts as well. A slow pupil response to the light or the lack of constriction of the pupil without the light directly shown on it indicates failure of the assessment.

1. An ocular motor assessment tool comprising:
a graduated, elongated shaft comprising a proximal end configured to be placed into contact with a nose of a patient;
a slide member that is adapted to slide along at least a portion of the length of the elongated shaft, the position of the slide member on the shaft being recorded by the location of the slide member relative to the shaft graduations; and
a tab that is connected to and slidable with the slide member, the tab being configured to extend upwardly from the slide member, when the contoured surface is placed in contact with the patient's nose and the shaft extended horizontally away from the patient's head the tab having a surface that is observable by the patient,
the tab surface comprising a first focal character located approximately level to the eyes of the patient.

2. The ocular motor assessment tool of claim 1, wherein the proximal end comprises a nose piece having a contoured surface that is configured to conform to the nose of the patient.

3. The ocular motor assessment tool of claim 2, wherein the nose piece includes a sidewall surface disposed laterally from the at least one contoured surface, the sidewall surface comprising a second focal character located thereon.

4. The ocular motor assessment tool of claim 3, wherein the slide member comprises a sidewall surface that comprises a third focal character located thereon, wherein the second and third focal characters are located on the same side of the tool and spaced apart from each other.

5. The ocular motor assessment tool of claim 2, wherein the nose piece contoured surface further comprises a recess inside of which a light is located.

6. The ocular motor assessment tool of claim 1, wherein the slide member comprises a window through which the shaft graduations can be viewed.

7. The ocular motor assessment tool of claim 1, wherein the tool is collapsible into at least two segments.

8. The ocular motor assessment tool of claim 7, wherein the at least two segments are connected by a tether.

9. The ocular motor assessment tool of claim 7, wherein the tool is shiftable between a collapsed configuration and an extended configuration.

10. The ocular motor assessment tool of claim 9, wherein in the collapsed configuration, the slide member is positioned adjacent to the proximal end.

11. The ocular motor assessment tool of claim 10, wherein the slide member is configured to engage and interlock with the proximal end to inhibit relative movement between the at least two segments.

12. The ocular motor assessment tool of claim 9, wherein the tab is configured to extend upwardly from the slide member when the tool is in the extended configuration and a stored configuration in which the tab extends downwardly toward the proximal end when the tool is in the collapsed configuration.

13. The ocular motor assessment tool of claim 7, wherein the at least two segments are configured such that one of the segments can be telescopically received within another of the segments.

14. The ocular motor assessment tool of claim 1, wherein the tool is configured for use in performing one or more eye movement assessments selected from the group consisting of a smooth pursuit assessment, a convergence assessment, a saccades assessment, and a vestibular ocular reflex assessment.

15. A method of ocular motor screening of a patient comprising:
providing the ocular motor assessment tool according to claim 1; and
performing one or more eye movement assessments with the ocular motor assessment tool selected from the group consisting of a smooth pursuit assessment, a convergence assessment, a saccades assessment, and a vestibular ocular reflex assessment.

16. The method of claim 1, wherein the ocular motor assessment tool is collapsible, and wherein the method further comprises extending the ocular motor assessment tool from a collapsed configuration to an extended configuration prior to performing the one or more eye movement assessments.

17. An ocular motor assessment tool comprising:
a graduated, elongated shaft comprising a proximal end configured to be placed into contact with a nose of a patient and a distal end;
a light assembly comprising a light source that is operable to emit a beam of light from the elongated shaft; and
a slide member that is adapted to slide along at least a portion of the length of the elongated shaft, the position of the slide member on the shaft being recorded by the location of the slide member relative to the shaft graduations.

18. The ocular motor assessment tool of claim 17, wherein the proximal end comprises a contoured surface that is configured to conform to the nose of the patient, the light source being located inside a recess formed in the contoured surface.

19. The ocular motor assessment tool of claim 17, wherein the light assembly further comprises a light actuator button, a switch, a resistor, and one or more batteries, the light source being an LED light.

20. The ocular motor assessment tool of claim 19, wherein the light actuator button extends through a sidewall of the elongated shaft proximal end, the switch, resistor, and one or more batteries being located within the elongated shaft.
